(19) United States
(12) Patent Application Publication
(10) Pub. No.: US 2020/0255365 A1
(43) Pub. Date: Aug. 13, 2020

(54) PROCESS FOR MAKING CARBOXYLIC ACIDS

(71) Applicant: ExxonMobil Research and Engineering Company, Annandale, NJ (US)

(72) Inventors: Kun Wang, Bridgewater, NJ (US); Jonathan E. Mitchell, Easton, PA (US)

(21) Appl. No.: 16/743,200

(22) Filed: Jan. 15, 2020

Related U.S. Application Data
(60) Provisional application No. 62/802,755, filed on Feb. 8, 2019.

(51) Int. Cl. C07C 51/145

(52) U.S. Cl. CPC C07C 51/145; C07C 53/08

(57) ABSTRACT

An integrated process for the preparation of carboxylic acids using iso-paraffins is provided. The process includes oxidatively carbonylating a compound having a carbon-hydrogen bond with dialkyl peroxide, carbon monoxide and water. Concurrently, the iso-paraffin is converted to iso-alcohol. The process provides access to a wide range of useful carboxylic acids and operates under relatively mild conditions.

### CROSS-REFERENCE TO RELATED APPLICATION

This application claims the benefit of U.S. Provisional Application No. 62/802755, filed on Feb. 08, 2019, the entire contents of which are incorporated herein by reference.

### FIELD

The present disclosure relates to an integrated process for making carboxylic acids with concurrent light paraffin upgrading. The process comprises 1) air oxidation of an iso-paraffin to a mixture of alkyl hydroperoxide and alcohol; 2) conversion of the alkyl hydroperoxide and alcohol to dialkyl peroxide; 3) oxidative carbonylation of a compound comprising at least one carbon hydrogen bond, for example a paraffin, using the dialkyl peroxide and carbon monoxide in the presence of water and, optionally, a metal-containing reagent, to yield carboxylic acids, while the dialkyl peroxide is converted to a tertiary alcohol.

### BACKGROUND

Carboxylic acids such as acetic acid, propionic acids, butanoic acids, valeric acids, pivalic acid, acrylic acid, and methacrylic acid are valuable chemicals or building blocks for a variety of high performance materials. However, these acids are difficult to make, often involving complicated processes such as methanol carbonylation (acetic acid), olefin oxidation (low yield) or hydrocarboxylation (requiring pressure as high as 200 bar and hazardous materials such as Ni(CO)₄). Alternative routes using readily available starting materials (e.g., paraffins, carbon monoxide) under mild conditions are desired. Furthermore, abundant light paraffins (e.g., C₂-C₅ paraffins) as a result of fracking in the North America (NA) region creates opportunities for upgrading of these feedstocks. Accordingly, alternative processes to make carboxylic acids with concurrent upgrading of light paraffins to higher value molecules are attractive.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

### SUMMARY

Disclosed herein is a novel, integrated process to make carboxylic acids with concurrent light paraffin upgrading. The process comprises three major reaction steps: 1) air oxidation of an iso-paraffin to a mixture of alkyl hydroperoxide and alcohol; 2) conversion of the alkyl hydroperoxide and alcohol to dialkyl peroxide; 3) oxidative carbonylation of a compound comprising at least one carbon hydrogen bond, for example paraffins (alkanes) using the dialkyl peroxide and carbon monoxide (CO) in the presence of water and, optionally, a metal-containing reagent, to carboxylic acids, while the dialkyl peroxide is converted to a tertiary alcohol. The net reaction is oxidative carboxylation of the compound comprising at least one carbon hydrogen bond (for example paraffins) to carboxylic acids using iso-paraffin and oxygen, while the iso-paraffin is upgraded to a tertiary alcohol. The carboxylic acids are of higher value than the starting compound comprising at least one carbon hydrogen bond (for example paraffins) and the tertiary alcohol may be recovered as a chemical, or dehydrated to iso-olefin, converted to ether (with methanol or ethanol) or used directly as high-octane gasoline blend.

In one aspect the present disclosure provides a process for making carboxylic acids, said process comprising:
(a) oxidizing a first feed stream comprising one or more iso-paraffins to form alkyl hydroperoxides and first tertiary alcohols;
(b) catalytically converting the alkyl hydroperoxides and first tertiary alcohols to dialkyl peroxides; and
(c) oxidatively carbonylating a second feed stream comprising a compound comprising at least one carbon-hydrogen bond using the dialkyl peroxides as a radical initiator to afford carboxylic acids, while the dialkyl peroxides are converted to second tertiary alcohols.

In some embodiments the first feed stream comprises an iso-paraffin selected from the group consisting of iso-butane, iso-pentane, iso-hexane, iso-heptane and mixtures thereof. In some preferred embodiments the first feed stream comprises iso-butane and the first tertiary alcohol is t-butyl alcohol.

In some embodiments the second feed stream comprises a compound selected from the group consisting of paraffins, cycloalkanes, alkenes, alkynes, aromatic hydrocarbons, heteroaromatic compounds, and mixtures thereof.

In one preferred embodiment the second feed stream comprises paraffins. Particularly preferred paraffins may be selected from the group consisting of methane, ethane, propane, butanes and mixtures thereof.

When the second feed stream comprises paraffins, oxidative carbonylation yields aliphatic carboxylic acids. For example when the second feed stream comprises propane the product of oxidative carbonylation is n-butanoic acid and/or isomers such as 2-methyl propanoic acid. Oxidative carbonylation of higher paraffinic homologues provides higher carboxylic acids, for example, pentanoic acid.

In another preferred embodiment the second feed stream comprises cycloalkanes. Particularly preferred cycloalkanes may be selected from the group consisting of cyclobutane, cyclopentane, cyclohexane, cycloheptane, cyclooctane and mixtures thereof. The cycloalkanes may be unsubstituted or substituted. Where the cycloalkanes are substituted, preferred substituents include one or more alkyl groups.

Where the second feed stream comprises cycloalkanes, oxidative carbonylation yields cycloalkane carboxylic acids. For example where the second feed stream comprises cyclohexane the product of oxidative carbonylation is cyclohexane carboxylic acid.

In another preferred embodiments the second feed stream comprises an alkene. Particularly preferred alkenes may be selected from the group consisting of ethylene, propene, butenes and mixtures thereof.

Where the second feed stream comprises an alkene, oxidative carbonylation yields unsaturated carboxylic acids comprising a carbon-carbon double bond. For example where the second feed stream comprises ethylene the product of oxidative carbonylation is acrylic acid.

In another preferred embodiment the second feed stream comprises an alkyne. Particularly preferred alkynes may be selected from the group consisting of acetylene, methylacetylene, butynes and mixtures thereof.

Where the second feed stream comprises an alkyne, oxidative carbonylation yields unsaturated carboxylic acids comprising a carbon-carbon triple bond. For example where the second feed stream comprises acetylene the product of oxidative carbonylation is propiolic acid.

In another preferred embodiment the second feed stream comprises an aromatic hydrocarbon. Particularly preferred aromatic hydrocarbons may be selected from the group consisting of benzene, alkylbenzenes such as toluene, ethylbenzene, cumene and xylenes, naphthalenes and mixtures thereof. The aromatic hydrocarbon may be substituted or unsubstituted. Where substituted the aromatic hydrocarbon may be substituted with one or more alkyl groups.

Where the second feed stream comprises an aromatic hydrocarbon, oxidative carbonylation yields aromatic carboxylic acids. For example where the second feed stream comprises benzene the product of oxidative carbonylation is benzoic acid.

In another preferred embodiment the second feed stream comprises a heteroaromatic compound.

Preferred carboxylic acids that may be prepared by the presently disclosed integrated process include acetic acid, propanoic acid, butanoic acids, valeric acids, pivalic acid, acrylic acid and methacrylic acid.

The presently disclosed process may offer one or more of the following advantages:
a wide range of useful carboxylic acids may be prepared
the process operates under relatively mild conditions
expensive and often toxic transition metal based catalysts may be avoided.

In some preferred embodiments the compound comprising at least one carbon-hydrogen bond comprises at least one aliphatic carbon-hydrogen bond capable of forming a radical with dialkylperoxide.

In other embodiments the compound comprising at least one carbon-hydrogen bond comprises one or more functional groups. The one or more functional groups may be selected from the group consisting of halogen, hydroxyl, cyano, carbonyl, carboxyl, amino, mercapto, nitro, sulfonato, phosphate, borato and combinations thereof.

The use of functionalized compounds provides the opportunity to synthesize a wide range of functionalized carboxylic acids.

The oxidative carbonylation is performed in the presence of water. The molar ratio of water to the compound comprising at least one carbon-hydrogen bond may be from about 0.05 to about 50, preferably about 0.05 to about 20, more preferably about 0.05 to about 5.

In some embodiments the oxidative carbonylation may be performed in the presence of one or more metal-containing reagents. Said metal-containing reagents may comprise a single metal reagent or a mixture of metal reagents. The metal-containing reagents may have variable oxidation state and belong to Group 3-16 of the Periodic Table of the Elements.

In some preferred embodiments the one or more metal-containing reagents is selected from the group consisting of Sc, Y, La, Ti, Zr, Hf, V, Nb, Ta, Cr Mo, W, Mn, Re, Fe, Ru, Co, Rh, Ir, Ni, Pd, Pt, Cu, Ag, Au, Zn, Ga, In, Ge, Sn, Sb, Bi, Te and rare earth complexes, salts thereof and mixtures thereof.

More preferably the one or more metal-containing reagents is selected from the group consisting of Ti, V, Nb, Cr, Mo, Mn, Fe, Co, Ni, Cu, Sn, and Bi complexes, salts thereof and mixtures thereof.

Even more preferably the one or more metal-containing reagents is selected from the group consisting of V, Nb, Mo, Mn, Fe, Co, Cu, Sn, and Bi complexes, salts thereof and mixtures thereof.

In some embodiments the molar ratio of the one or more metal-containing reagents to the compound comprising at least one carbon-hydrogen bond is from about 0.001 to about 0.5, preferably from about 0.005 to about 0.5, more preferably from about 0.01 to about 0.5.

Further features and advantages of the present disclosure will be understood by reference to the following drawings and detailed description.

### BRIEF DESCRIPTION OF THE DRAWINGS

FIG. 1 is a flow scheme of a process according to one embodiment of the present disclosure.

### DETAILED DESCRIPTION OF THE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure.

Although any methods and materials similar or equivalent to those described herein can also be used in the practice or testing of the present disclosure, the preferred methods and materials are now described.

It must also be noted that, as used in the specification and the appended claims, the singular forms ‘a’, ‘an’ and ‘the’ include plural referents unless otherwise specified. Thus, for example, reference to ‘paraffin’ may include more than one paraffins, and the like.

Throughout this specification, use of the terms ‘comprises’ or ‘comprising’ or grammatical variations thereon shall be taken to specify the presence of stated features, integers, steps or components but does not preclude the presence or addition of one or more other features, integers, steps, components or groups thereof not specifically mentioned.

Unless specifically stated or obvious from context, as used herein, the term “about” is understood as within a range of normal tolerance in the art, for example within two standard deviations of the mean. ‘About’ can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein in the specification and the claim can be modified by the term ‘about’.

Any processes provided herein can be combined with one or more of any of the other processes provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The chemistry of Steps 1-3 with respect to iso-butane feed is shown below in corresponding reactions 1-3:

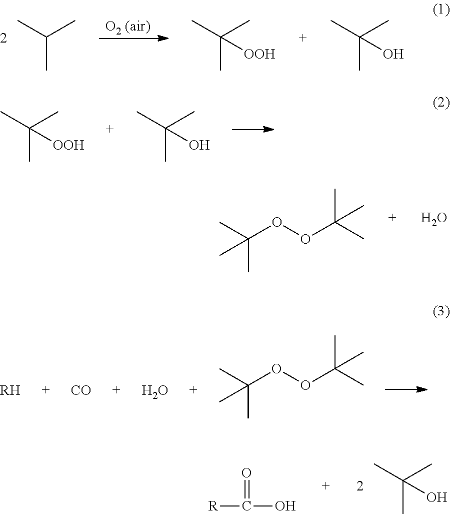

Reaction 1 (Step 1) represents the air oxidation of iso-butane to t-butylhydroperoxide and t-butyl alcohol.

Reaction 2 (Step 2) illustrates conversion of t-butylhydroperoxide and t-butyl alcohol to di-t-butyl peroxide (DTBP) using an acid catalyst.

Reaction 3 (Step 3) illustrates the oxidative carbonylation of a compound comprising a carbon-hydrogen bond (RH) using DTBP. The DTBP is converted into t-butyl alcohol. The role of DTBP may be to initiate an organic radical (R.). The radical may then add to carbon monoxide to give an acyl radical [RC(O).] which is then converted to carboxylic acid in the presence of water. In some embodiments a metal-containing reagent may be used in the oxidative carbonylation step to facilitate the reaction. Said metal-containing reagent comprises a metal or mixture of metals with variable oxidation state in the form of salt or complex belonging to Group 3-16 in the Periodic Table of the Elements.

Steps 1 and 2 have been previously described in applicant's co-pending application, U.S. App. Publ. No. 2017/0101366, incorporated by reference herein in its entirety. U.S. App. Publ. No. 2017/0101366 describes a process to couple functional molecules into di-functional or multi-functional molecules using dialkyl peroxide as a radical initiator. Whereas U.S. App. Publ. No. 2017/0101366 is directed to create di-functional or multi-functional molecules utilizing coupling reactions, the present disclosure utilizes dialkyl peroxide to initiate oxidative carbonylation of, for example, a paraffin, to yield carboxylic acids.

Iso-butane oxidation in Step 1/Reaction 1 is commercially well-established for making t-butyl hydroperoxide (TBHP) for propylene oxide manufacture. Variants of the process are described, for example, in U.S. Pat. No. 2,845,461; U.S. Pat. No. 3,478,108; U.S. Pat. No. 4,408,081 and U.S. Pat. No. 5,149,885. EP 0567336 and U.S. Pat. No. 5,162,593 disclose co-production of TBHP and t-butyl alcohol (TBA).

As TBA is one of the reactants used in Step 2 of the present disclosure, the present integrated process scheme utilizes Step 1 as a practical source of these two reactants. Air (approximately 21% oxygen), a mixture of nitrogen and oxygen containing 2-20 vol % oxygen, or pure oxygen, can be used for the oxidation, as long as the oxygen-to-hydrocarbon vapor ratio is kept outside the explosive regime. Preferably air is used as the source of oxygen.

Step 1/Reaction 1 is preferably carried out at a temperature from about 110 to about 150° C., more preferably from about 130 to about 140° C.

The pressure is preferably from about 300 to about 800 psig, more preferably from about 450 to about 550 psig.

The reaction time may be from about 2 hours to about 24 hours, preferably from about 6 hours to about 8 hours. Such reaction times typically produce conversions from about 15% to about 70%, preferably from about 30 to about 50%.

Typically, selectivity to TBHP is from about 50 to about 80%, and to TBA from about 20 to about 50%.

In Step 2/Reaction 2, the conversion of the TBHP and TBA to di-t-butyl peroxide (DTBP) is performed using an acid catalyst. For example, U.S. Pat. No. 5,288,919 describes the use of an inorganic heteropoly and/or isopoly acid catalyst (such as for the reaction of TBA with TBHP). The concurrent production of DTBP and TBA from TBHP is also described in U.S. Pat. No. 5,345,009.

A preferred configuration for Step 2 of the presently disclosed integrated process uses reactive distillation in which product water is continuously removed as an overhead by-product.

Step 2 is preferably carried out at a temperature from about 50 to about 200° C., more preferably from about 60 to about 150° C., even more preferably from about 80 to about 120° C.

Pressure for the reaction is held within appropriate ranges to ensure the reaction occurs substantially in the liquid phase, for example, from about 0 to about 300 psig, preferably from about 5 to about 100 psig, more preferably from about 15 to about 50 psig.

The TBHP to TBA mole ratio may be in the range from about 0.5 to about 2, preferably from about 0.8 to about 1.5, more preferably from about 0.9 to about 1.1. The reaction may be performed with or without a solvent. Suitable solvents comprise hydrocarbons having a carbon number greater than 3. Suitable solvents include paraffins, cycloalkanes, or aromatics.

Advantageously, the unreacted iso-butane from Step 1 may be used as a solvent for Step 2.

An acid catalyst such as Amberlyst™ resin, Nafion™ resin, aluminosilicates, acidic clay, zeolites (natural or synthetic), silicoaluminophosphates (SAPO), heteropolyacids, acidic oxides such as tungsten oxide on zirconia, molybdenum oxide on zirconia, sulfonated zirconia, liquid acids such sulfuric acid, or acidic ionic liquids may be used in Step 2/Equation 2 to promote the conversion of TBHP and TBA into DTBP.

Reaction (3) is preferably carried out at a temperature from about 100 and about 170° C., more preferably from about 130 and about 150° C. The pressure is preferably from about 100 and about 3000 psig, more preferably from about 500 and 3000 psig. The CO pressure is preferably from about 300 and about 3000 psig, more preferably from about 500 to about 3000 psig. The amount of water, defined as the mole ratio of H₂O to RH is preferably from about 0.05 to about 50, more preferably from about 0.05 to about 20, and most preferably from about 0.05 to about 5.

In some embodiments one or more metal-containing reagents may be used in the oxidative carbonylation step to facilitate the reaction. Said metal-containing reagents may comprise a metal or mixture of metals with variable oxidation state in the form of salt or complex belonging to Group 3-16 in the Periodic Table of the Elements. Examples of the metal-containing reagents include, but are not limited to, salts and/or complexes of Sc, Y, La, rare-earth metals, Ti, Zr, Hf, V, Nb, Ta, Cr Mo, W, Mn, Re, Fe, Ru, Co, Rh, Ir, Ni, Pd, Pt, Cu, Ag, Au, Zn, Ga, In, Ge, Sn, Sb, Bi, and Te. Preferably the metal-containing reagent is a salt and/or complex of Ti, V, Nb, Cr, Mo, Mn, Fe, Co, Ni, Cu, Sn, and Bi. More preferably the metal-containing reagent is a salt and/or complex of V, Nb, Mo, Mn, Fe, Co, Cu, Sn, and Bi. The molar ratio of the one or more metal-containing reagent to the compound comprising at least one carbon-hydrogen bond may be from about 0.001 to about 0.5, preferably from about 0.005 to about 0.5, more preferably from about 0.01 to about 0.5.

The reaction time may be from about 2 hours to about 24 hours, preferably from about 4 hours to about 16 hours. Typical conversions may be from about 15 to about 70%, preferably about 40%.

Complete conversion of DTBP is typically achieved in Reaction (3).

The compound (RH) comprising a carbon-hydrogen bond in Reaction (3) can be any paraffin, cycloalkane, olefin, alkyne, aromatic hydrocarbon, or heteroaromatic compound.

Examples of paraffins comprise methane, ethane, propane, or C4+ alkanes. Examples of cycloalkanes include cyclobutane, cyclopentane, cyclohexane, cycloheptane, cyclooctane, either substituted with unsubstituted with alkyl groups. Examples of olefins include ethylene, propylene, or C4+ olefins. Examples of alkynes include acetylene, methylacetylene, or C4+ alkynes. Examples of aromatic hydrocarbons include benzene, alkylbenzene such as toluene, ethylbenzene, cumene, xylenes; naphthalenes (either substituted or unsubstituted).

RH in Reaction (3) can also be a functional molecule containing at least one aliphatic C—H bond that can form a radical in the presence of DTBP. Examples of functional groups comprise halogen, —OH (hydroxyl), —CN (cyano), —C(O)OH (carboxylic), —NHR′ (amino, where R′ can be H or a hydrocarbyl group), —SH (mercapto), —NO₂ (nitro), —OSO₃H (sulfonato), —OPO₃H (phosphato), —OBO (borato), and the like.

The overall reaction stoichiometry from equations 1 to 3 is shown below in Equation 4.

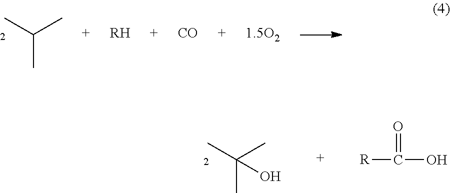

The net effect of Equations 1 to 3 is oxidative carbonylation of R-H to carboxylic acid using iso-butane as an oxygen carrier, while iso-butane is converted to t-butyl alcohol—an upgraded product from the iso-paraffin iso-butane. Depending on the nature of the iso-paraffin, the resulting alcohol can be used as high octane blend for gasoline: e.g., t-butyl alcohol from iso-butane, or 2-methyl-2-butanol from iso-pentane. Alternatively, the alcohols can be converted to olefins as chemical products via dehydration (e.g., iso-butylene), or etherified with an alcohol such as methanol or ethanol making ether as gasoline blend (e.g., MTBE or ETBE from isobutane).

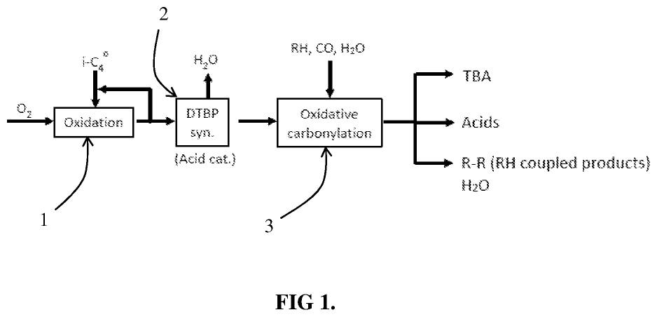

FIG. 1 illustrates a process scheme according to one embodiment of the present disclosure in which iso-butane is the feed for the initial oxidation step. A feed comprising iso-butane (i-C₄°) is sent to an oxidation reactor (1) to which an oxidizing gas comprising O₂ is also fed.

The oxidation mixture comprising t-butyl hydroperoxide (TBHP) and t-butyl alcohol (TBA) is sent to the next reactor (2) after i-C4° is separated and optionally recycled to the oxidation reactor (1), where di-t-butyl peroxide (DTBP) is formed over an acid catalyst (for example Amberlyst or acidic clay). A preferred configuration for this reactor is reactive distillation where the co-product water is continuously removed (as illustrated).

DTBP is sent to the next reactor (3) to initiate carbonylation of feed RH.

The reaction products are separated/fractionated in the next step. Unreacted RH and CO are recycled to the carbonylation reactor (3). Final products from this process include carboxylic acids as a result of the carbonylation of RH, t-butyl alcohol (TBA), by-product acetone and heavier products as a result of RH oxidative coupling (e.g., R-R). Excess water, if any, can either be recycled to the carbonylation reactor (3) or discharged after appropriate treatment.

Reference will now be made to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

### EXAMPLE 1

### Formation of Butanoic Acids from Propane, CO, and H₂O in the Presence of DTBP

In a 300-cc autoclave 6 g of water (0.33 mole) and 24 g of di-t-butyl peroxide (DTBP, under the trade name Luperox DI from Aldrich Chemicals, 98%) were loaded. The autoclave was sealed, connected to a LPG manifold, and charged with 100 cc of liquid propane (1.32 mole). The autoclave was then pressurized with 600 psig of CO, and the contents heated under stirring (800 rpm) at a rate of 2° C./min to 135° C. and held for 21 h. The heat was turned off and the autoclave allowed to cool down to room temperature and the gaseous components vented. The autoclave was opened, the reactor contents were collected, and 10 g of weight gain was registered. The products were analyzed by GC and GC/MS which indicated the formation of acetic acid (˜30 wt %, side product from methyl radical as a result of β-scission of t-butoxy radical) and C4 carboxylic acids [n-butanoic acid (˜10 wt %), and 2-methyl propanoic acid (˜5 wt %), which are expected products from propane oxidative carbonylation] together with C6+ hydrocarbons (˜40 wt %) as a result of propane oxidative coupling.

### EXAMPLE 2

### Formation of Cyclohexane Carboxylic Acid from Cyclohexane, CO, and H₂O in the Presence of DTBP

In a 300-cc autoclave 6 g of water (0.33 mole), 24 g of di-t-butyl peroxide (DTBP, under the trade name Luperox DI from Aldrich Chemicals, 98%), and 78 g of cyclohexane (0.93 mole) were loaded. The autoclave was sealed and pressurized with 600 psig of CO, and the contents heated under stirring (800 rpm) at a rate of 2° C./min to 135° C. and held for 21 h. The heat was turned off, and the autoclave allowed to cool down to room temperature and the gaseous components vented. The autoclave was opened, and the reactor contents collected. The products were analyzed by GC and GC/MS which indicated the formation of acetic acid (˜20 wt %, side product from methyl radical as a result of β-scission of t-butoxy radical) and cyclohexane carboxylic acid (˜5 wt %, which is the expected product from cyclohexane oxidative carbonylation) together with C12+ hydrocarbons (˜75 wt %) as a result of cyclohexane oxidative coupling.

### EXAMPLE 3

### Formation of Cyclohexane Carboxylic Acid from Cyclohexane, CO, and H₂O in the Presence of DTBP

In a 300-cc autoclave, 45 g of water (2.5 mole), 24 g of di-t-butyl peroxide (DTBP, under the trade name Luperox DI from Aldrich Chemicals, 98%) and 78 g of cyclohexane (0.93 mole) were loaded. The autoclave was sealed and pressurized with 600 psig of CO, and the contents heated under stirring (800 rpm) at a rate of 2° C./min to 135° C. and held for 21 h. The heat was turned off and the autoclave allowed to cool down to room temperature, and gaseous components vented. The autoclave was opened, and the reactor contents collected. The products were analyzed by GC and GC/MS which indicated the formation of acetic acid, cyclohexane carboxylic acid (expected product from cyclohexane oxidative carbonylation together with C12+ hydrocarbons as a result of cyclohexane oxidative coupling.

### Certain Embodiments

Certain embodiments of processes according to the present disclosure are presented in the following paragraphs.

Embodiment 1 provides a process for making carboxylic acids, said process comprising:
(a) oxidizing a first feed stream comprising one or more iso-paraffins to form alkyl hydroperoxides and first tertiary alcohols;
(b) catalytically converting the alkyl hydroperoxides and first tertiary alcohols to dialkyl peroxides; and
(c) oxidatively carbonylating a second feed stream comprising a compound comprising at least one carbon-hydrogen bond using the dialkyl peroxides as a radical initiator to afford carboxylic acids, while the dialkyl peroxides are converted to second tertiary alcohols.

Embodiment 2 provides a process according to Embodiment 1, wherein the first feed stream comprises an iso-paraffin selected from the group consisting of iso-butane, iso-pentane, iso-hexane, iso-heptane and mixtures thereof.

Embodiment 3 provides a process according to Embodiment 1 or Embodiment 2, wherein the first feed stream comprises iso-butane.

Embodiment 4 provides a process according to any one of Embodiments 1 to 3, wherein the second feed stream comprises a compound selected from the group consisting of paraffins, cycloalkanes, alkenes, alkynes, aromatic hydrocarbons, heteroaromatic compounds, and mixtures thereof.

Embodiment 5 provides a process according to any one of Embodiments 1 to 4, wherein the second feed stream comprises paraffins.

Embodiment 6 provides a process according to any one of Embodiments 1 to 5, wherein the second feed stream comprises a paraffin selected from the group consisting of methane, ethane, propane, butanes and mixtures thereof.

Embodiment 7 provides a process according to any one of Embodiments 1 to 4, wherein the second feed stream comprises cycloalkanes.

Embodiment 8 provides a process according to any one of claims 1 to 4, wherein the second feed stream comprises a cycloalkane selected from the group consisting of cyclobutane, cyclopentane, cyclohexane, cycloheptane, cyclooctane and mixtures thereof.

Embodiment 9 provides a process according to Embodiment 8, wherein the cycloalkanes are substituted with one or more alkyl groups.

Embodiment 10 provides a process according to any one of Embodiments 1 to 4, wherein the second feed stream comprises an alkene.

Embodiment 11 provides a process according to any one of Embodiments 1 to 4, wherein the second feed stream comprises an alkene selected from the group consisting of ethylene, propene, butenes and mixtures thereof.

Embodiment 12 provides a process according to any one of Embodiments 1 to 4, wherein the second feed stream comprises an alkyne.

Embodiment 13 provides a process according to Embodiment 12, wherein the alkyne is selected from the group consisting of acetylene, methylacetylene, butynes and mixtures thereof.

Embodiment 14 provides a process according to any one of Embodiments 1 to 4, wherein the second feed stream comprises an aromatic hydrocarbon.

Embodiment 15 provides a process according to Embodiment 14, wherein the aromatic hydrocarbon is substituted with one or more alkyl groups.

Embodiment 16 provides a process according to Embodiment 14, wherein the aromatic hydrocarbon is selected from the group consisting of benzene, alkylbenzenes such as toluene, ethylbenzene, cumene and xylenes, naphthalenes and mixtures thereof.

Embodiment 17 provides a process according to any one of Embodiments 1 to 4, wherein the second feed stream comprises a heteroaromatic compound.

Embodiment 18 provides a process according to any one of Embodiments 1 to 17, wherein the compound comprising at least one carbon-hydrogen bond comprises at least one aliphatic carbon-hydrogen bond capable of forming a radical with dialkylperoxide.

Embodiment 19 provides a process according to any one of Embodiments 1 to 18, wherein the compound comprising at least one carbon-hydrogen bond comprises one or more further functional groups.

Embodiment 20 provides a process according to Embodiment 19, wherein the one or more further functional groups are selected from the group consisting of halogen, hydroxyl, cyano, carbonyl, carboxyl, amino, mercapto, nitro, sulfonato, phosphate, borato and combinations thereof.

Embodiment 21 provides a process according any one of Embodiments 1 to 20, wherein the carboxylic acid is selected from the group consisting of acetic acid, propanoic acid, butanoic acids, valeric acids, pivalic acid, acrylic acid and methacrylic acid.

Embodiment 22 provides a process according to any one of Embodiments 1 to 21, wherein the oxidative carbonylation is performed in the presence of water.

Embodiment 23 provides a process according to Embodiment 22, wherein the molar ratio of water to the compound comprising at least one carbon-hydrogen bond is from about 0.05 to about 50, preferably about 0.05 to about 20, more preferably about 0.05 to about 5.

Embodiment 24 provides a process according to any one of Embodiments 1 to 23, wherein the oxidative carbonylation is performed in the presence of one or more metal-containing reagents.

Embodiment 25 provides a process according to Embodiment 24, wherein the one or more metal-containing reagents is selected from the group consisting of Sc, Y, La, Ti, Zr, Hf, V, Nb, Ta, Cr Mo, W, Mn, Re, Fe, Ru, Co, Rh, Ir, Ni, Pd, Pt, Cu, Ag, Au, Zn, Ga, In, Ge, Sn, Sb, Bi, Te and rare earth complexes, salts thereof and mixtures thereof.

Embodiment 26 provides a process according to Embodiment 24 or Embodiment 25, wherein the one or more metal-containing reagent is selected from the group consisting of Ti, V, Nb, Cr, Mo, Mn, Fe, Co, Ni, Cu, Sn, and Bi complexes, salts thereof and mixtures thereof.

Embodiment 27 provides a process according to any one of Embodiments 24 to 26, wherein the one or more metal-containing reagent is selected from the group consisting of V, Nb, Mo, Mn, Fe, Co, Cu, Sn, and Bi complexes, salts thereof and mixtures thereof.

Embodiment 28 provides a process according to any one of Embodiments 24 to 27, wherein the molar ratio of the one or more metal-containing reagents to the compound comprising at least one carbon-hydrogen bond is from about 0.001 to about 0.5, preferably from about 0.005 to about 0.5, more preferably from about 0.01 to about 0.5.

The contents of all references, including published patents and patent applications cited throughout the application are hereby incorporated by reference.

It is understood that the detailed examples and embodiments described herein are given

by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

1. A process for making carboxylic acids, said process comprising:
(a) oxidizing a first feed stream comprising one or more iso-paraffins to form alkyl hydroperoxides and first tertiary alcohols;
(b) catalytically converting the alkyl hydroperoxides and first tertiary alcohols to dialkyl peroxides; and
(c) oxidatively carbonylating a second feed stream comprising a compound comprising at least one carbon-hydrogen bond using the dialkyl peroxides as a radical initiator to afford carboxylic acids, while the dialkyl peroxides are converted to second tertiary alcohols.

2. A process according to claim 1, wherein the first feed stream comprises an iso-paraffin selected from the group consisting of iso-butane, iso-pentane, iso-hexane, iso-heptane and mixtures thereof.

3. A process according to claim 1, wherein the first feed stream comprises iso-butane.

4. A process according to claim 1, wherein the second feed stream comprises a compound selected from the group consisting of paraffins, cycloalkanes, alkenes, alkynes, aromatic hydrocarbons, heteroaromatic compounds, and mixtures thereof.

5. A process according to claim 4, wherein the second feed stream comprises a paraffin.

6. A process according to claim 4, wherein the second feed stream comprises a paraffin selected from the group consisting of methane, ethane, propane, butanes and mixtures thereof.

7. A process according to claim 4, wherein the second feed stream comprises a cycloalkane.

8. A process according to claim 4, wherein the second feed stream comprises a cycloalkane selected from the group consisting of cyclobutane, cyclopentane, cyclohexane, cycloheptane, cyclooctane and mixtures thereof.

9. A process according to claim 4, wherein the cycloalkanes are substituted with one or more alkyl groups.

10. A process according to claim 4, wherein the second feed stream comprises an alkene.

11. A process according to claim 4, wherein the second feed stream comprises an alkene selected from the group consisting of ethylene, propene, butenes and mixtures thereof.

12. A process according to claim 4, wherein the second feed stream comprises an alkyne.

13. A process according to claim 4, wherein the alkyne is selected from the group consisting of acetylene, methylacetylene, butynes and mixtures thereof.

14. A process according to claim 4, wherein the second feed stream comprises an aromatic hydrocarbon.

15. A process according to claim 4, wherein the aromatic hydrocarbon is substituted with one or more alkyl groups.

16. A process according to claim 4, wherein the aromatic hydrocarbon is selected from the group consisting of benzene, alkylbenzenes such as toluene, ethylbenzene, cumene and xylenes, naphthalenes and mixtures thereof.

17. A process according to claim 4, wherein the second feed stream comprises a heteroaromatic compound.

18. A process according to claim 1, wherein the compound comprising at least one carbon-hydrogen bond comprises at least one aliphatic carbon-hydrogen bond capable of forming a radical with dialkylperoxide.

19. A process according to claim 1, wherein the compound comprising at least one carbon-hydrogen bond comprises one or more further functional groups.

20. A process according to claim 19, wherein the one or more further functional groups are selected from the group consisting of halogen, hydroxyl, cyano, carbonyl, carboxyl, amino, mercapto, nitro, sulfonato, phosphate, borato and combinations thereof.

21. A process according to claim 1, wherein the carboxylic acid is selected from the group consisting of acetic acid, propanoic acid, butanoic acids, valeric acids, pivalic acid, acrylic acid and methacrylic acid.

22. A process according to claim 1, wherein the oxidative carbonylation is performed in the presence of water.

23. A process according to claim 22, wherein the molar ratio of water to the compound comprising at least one carbon-hydrogen bond is from about 0.05 to about 50, preferably about 0.05 to about 20, more preferably about 0.05 to about 5.

24. A process according to claim 1, wherein the oxidative carbonylation is performed in the presence of one or more metal-containing reagents.

25. A process according to claim 24, wherein the one or more metal-containing reagents is selected from the group consisting of Sc, Y, La, Ti, Zr, Hf, V, Nb, Ta, Cr Mo, W, Mn, Re, Fe, Ru, Co, Rh, Ir, Ni, Pd, Pt, Cu, Ag, Au, Zn, Ga, In, Ge, Sn, Sb, Bi, Te and rare earth complexes, salts thereof and mixtures thereof.

26. A process according to claim 24, wherein the one or more metal-containing reagents is selected from the group consisting of Ti, V, Nb, Cr, Mo, Mn, Fe, Co, Ni, Cu, Sn, and Bi complexes, salts thereof and mixtures thereof.

27. A process according to claim 24, wherein the one or more metal-containing reagent is selected from the group consisting of V, Nb, Mo, Mn, Fe, Co, Cu, Sn, and Bi complexes, salts thereof and mixtures thereof.

28. A process according to claim 24, wherein the molar ratio of the one or more metal-containing reagents to the compound comprising at least one carbon-hydrogen bond is from about 0.001 to about 0.5, preferably from about 0.005 to about 0.5, more preferably from about 0.01 to about 0.5.
